(19) United States
(12) Patent Application Publication
(10) Pub. No.: US 2025/0255527 A1
(43) Pub. Date: Aug. 14, 2025

(54) NEURAL INTERFACE AND METHOD FOR MANUFACTURING SAME

(71) Applicant: DAEGU GYEONGBUK INSTITUTE OF SCIENCE AND TECHNOLOGY, Daegu (KR)

(72) Inventors: Sang Hoon LEE, Daegu (KR); Young Jun CHO, Daegu (KR); Hee Jae SHIN, Daegu (KR)

(21) Appl. No.: 18/845,576

(22) PCT Filed: Mar. 28, 2023
(86) PCT No.: PCT/KR2023/004092

§ 371 Date: Apr. 24, 2025

(30) Foreign Application Priority Data
Mar. 28, 2022 (KR) 10-2022-0038182

(51) Int. Cl. A61B 5/268; A61B 5/294

(52) U.S. Cl. CPC A61B 5/268; A61B 5/294; A61B 2562/125

(57) ABSTRACT

Disclosed herein are a neural interface and a method for fabricating the neural interface. The neural interface comprises: an electrode section for neural stimulation and neural signal recording; a first region defined by a first hole; and a second region defined by a second hole disposed within the first region, wherein the electrode section comprises a first electrode section and a second electrode section, either the first electrode section or the second electrode section being disposed within the second region.

The fabricating method comprises the steps of synthesizing a shape memory polymer; first coating a substrate with the shape memory polymer; forming a photoresist on the shape memory polymer; masking the photoresist; sputtering an electrode material; patterning the electrode material through a photolithography process; second coating the shape memory polymer; and etching the shape memory polymer.

### TECHNICAL FIELD

Various embodiments of the present disclosure relate to a neural interface and a fabricating method therefor and, more specifically, to a neural interface with a dual clip design utilizing shape memory polymers and a method for fabricating same.

### BACKGROUND ART

For use in implantable neural interfaces for recording and stimulating neural signals of peripheral nerves, it is very important to select materials that have biocompatibility to minimize immune response within the body, as well as softness similar to nerves. Also, the design and shape for maximizing the performance of insertion, recording, and stimulation of the neural interface are crucial as they directly the stability of the neural interface.

Shape memory polymers are frequently used as biomedical engineering materials due to their biocompatibility and shape memory effects. Previously, neural interfaces utilizing shape memory polymers have been developed, but they were used as additional substrates leveraging only the shape memory effect, separate from neural interfaces including conductive electrodes. Because of the use of relatively thick substrates, the overall neural interface becomes thicker, which reduces its stability for long-term use and makes it unsuitable for insertion into very small peripheral nerves.

Recently, there have been implementations of neural interfaces using shape memory polymers that are thinner and can be micro-patterned, allowing for the recording of more neural signal channels compared to conventional commercialized silicon cuff electrodes. However, the shape memory polymers used have a high glass transition temperature (66.3° C. in a dry state), resulting in insufficient softness at room or body temperature, and the high glass transition temperature limits the utilization of shape memory effects at physiological temperatures. Additionally, the use of E-Beam for depositing gold conductive layers in the process is costly, making it unsuitable for mass production.

### DISCLOSURE OF INVENTION

### Technical Problem

To address the problems encountered in the related art, the present disclosure is to provide a neural interface that includes a shape memory polymer exhibiting shape memory effects at physiological temperatures and a design that allows for dual clipping, and a fabricating method therefor.

### Solution to Problem

A neural interface according to various embodiments of the present disclosure includes: an electrode section for neural stimulation and neural signal recording; a first region defined by a first hole; and a second region defined by a second hole disposed within the first region, wherein the electrode section includes a first electrode section and a second electrode section, either the first electrode section or the second electrode section being disposed within the second region.

A method for fabricating a neural interface according to various embodiments of the present disclosure may include the steps of: synthesizing a shape memory polymer; first coating a substrate with the shape memory polymer; forming a photoresist on the shape memory polymer; masking the photoresist; sputtering an electrode material; patterning the electrode material through a photolithography process; second coating the shape memory polymer; and etching the shape memory polymer.

### Advantageous Effects of Invention

The neural interface of the present disclosure can be easily inserted into very small nerves due to the shape memory effect of the shape memory polymer. Additionally, the low glass transition temperature (44° C. in a dry state) enables morphological transformation through the shape memory effect without stressing the biological tissues, facilitating easy implantation of the neural interface and minimizing damage to the neural interface and the neural tissue. The neural interface also exhibits a Young's modulus of 400 MPa at 37° C., which is softer than the conventional polyimide (2.5 GPa) used for neural interface substrates.

The dual clip design of the neural interface allows the electrodes to contact both the upper and lower parts of the nerve, enabling stable fixation by double clipping the nerve.

In the method for fabricating a neural interface according to the present disclosure, sputtering and photolithography processes are used to directly deposit and pattern the conductive material, eliminating the need for pre-metal layer processes (stamping), which allows for fabrication with a single wafer. Additionally, using sputtering instead of the conventional E-beam process for metal deposition reduces process costs and makes the method more suitable for mass production.

### BRIEF DESCRIPTION OF DRAWINGS

FIG. 1 is a plan view of a neural interface according to an embodiment of the present disclosure.

FIG. 2 is a cross-sectional view taken along line I-I′ of FIG. 1.

FIG. 3 is a cross-sectional view taken along line II-II′ of FIG. 1.

FIG. 4 is a cross-sectional view taken along line III-III′ of FIG. 1.

FIG. 5 is a schematic diagram of the injection process of the neural interface according to an embodiment of the present disclosure.

FIG. 6 is a process flowchart of the method for fabricating the neural interface according to an embodiment of the present disclosure.

FIG. 7 is a schematic diagram of the fabricating process of the neural interface according to an embodiment of the present disclosure.

FIG. 8 shows (a) a graph of the storage modulus of the shape memory polymer and (b) a graph of the loss factor (tan δ).

FIG. 9 shows (a) impedance measurement results of the gold (Au) deposited neural interface and IrO₂ coated neural interface, and (b) CV test results.

FIG. 10 shows (a) a graph of the impedance and charge storage capacity (CSC) changes with applied voltage, (b) a CV curve with applied voltage over time, and (c) the bending process stability.

FIG. 11 shows (a) a schematic diagram explaining the implantation process of the neural interface, (b) a photograph of the neural interface implanted in the CPN, and (c) a photograph of the neural interface implanted in the PN.

FIG. 12 shows (a) a schematic diagram of the stimulation and recording process, (b) recorded EMG signals according to neural stimulation through the neural interface, (c) a bar graph of the peak-to-peak amplitude of the EMG signals, (d) recorded EMG signals according to neural stimulation through the hook electrode, and (e) a bar graph of the peak-to-peak amplitude of the CNAP.

### BEST MODE FOR CARRYING OUT THE INVENTION

Hereinafter, various embodiments of the present disclosure are described with reference to the accompanying drawings. The embodiments and the terminology used are not intended to limit the techniques described herein to specific forms of implementation, and it should be understood that various modifications, equivalents, and/or substitutions are included.

A detailed description will be given of the embodiments of the present disclosure with reference to the accompanying drawings as follows.

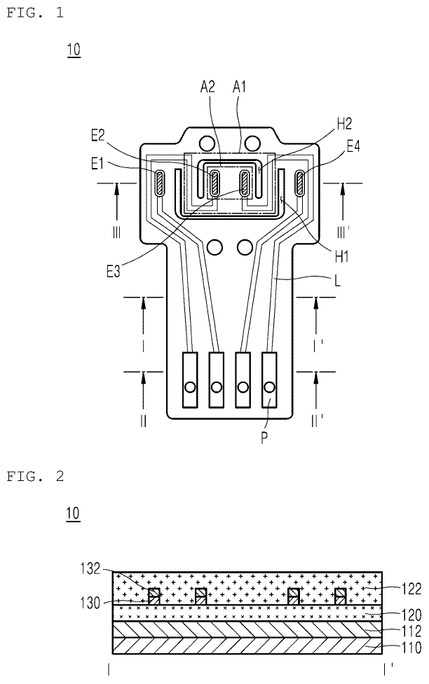

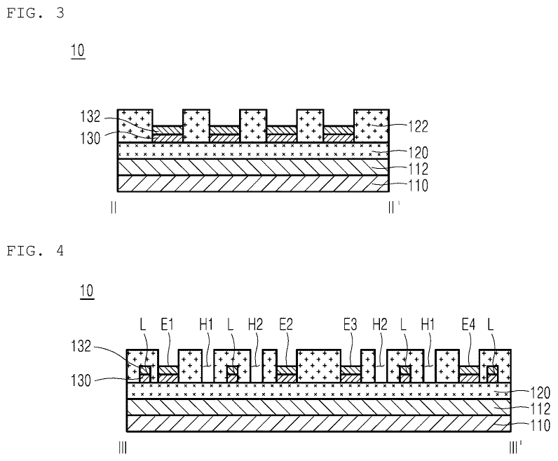

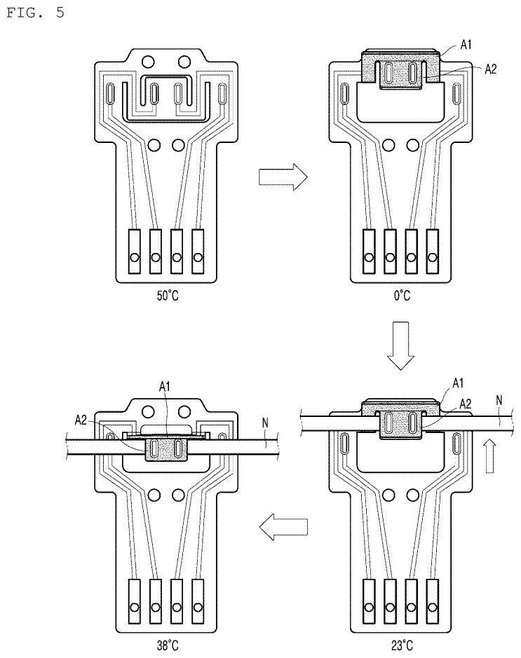

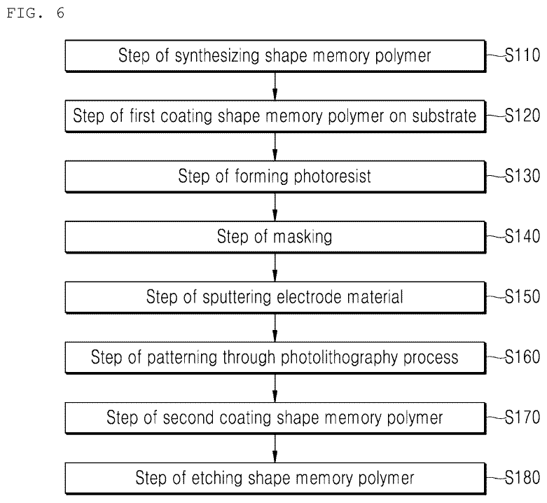

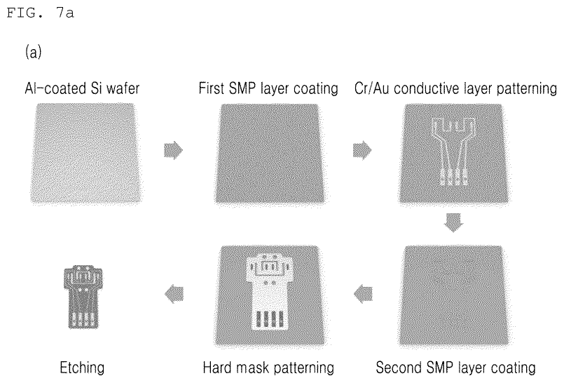

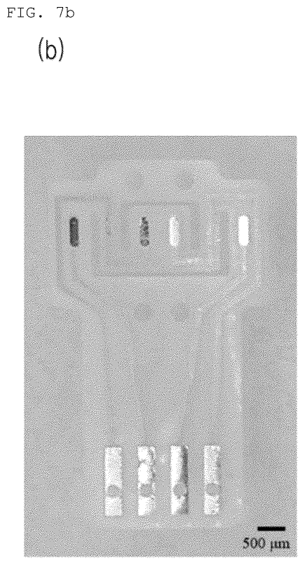

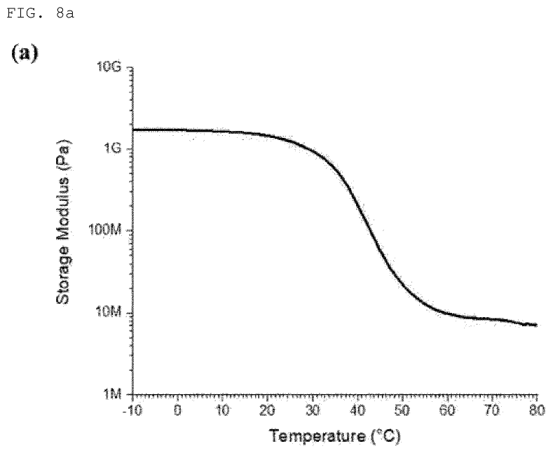

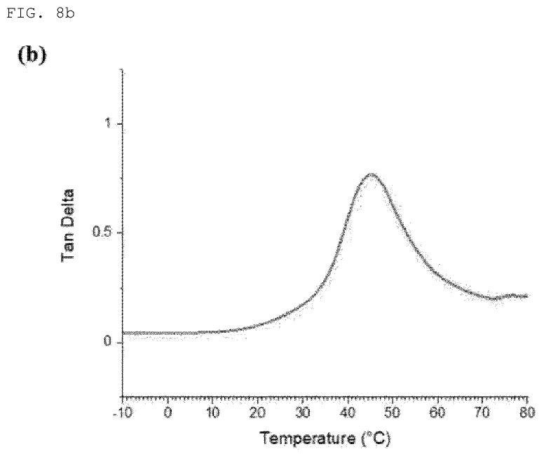

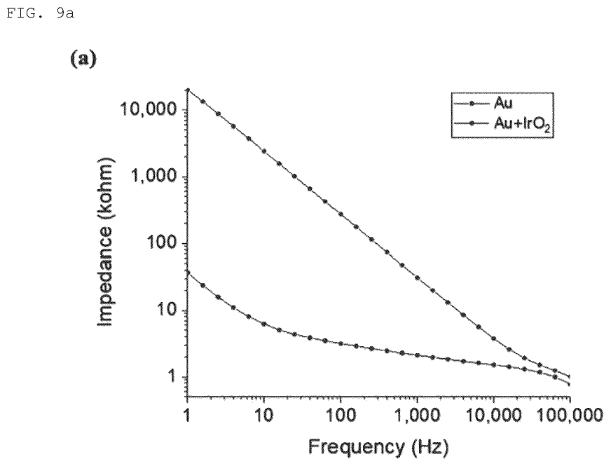

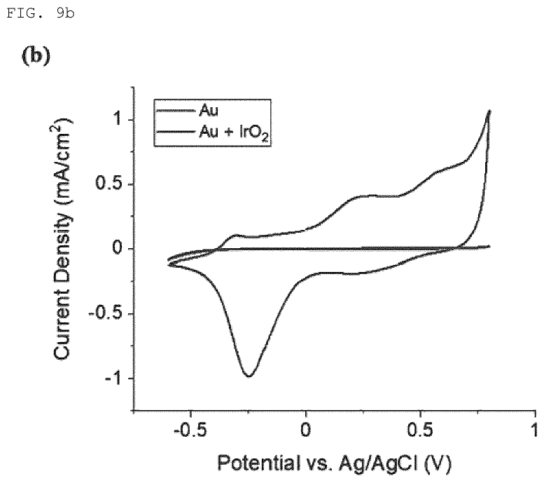

FIG. 1 is a plan view of a neural interface according to an embodiment of the present disclosure. FIG. 2 is a cross-sectional view taken along line I-I′ of FIG. 1. FIG. 3 is a cross-sectional view taken along line II-II′ of FIG. 1. FIG. 4 is a cross-sectional view taken along line III-III′ of FIG. 1.

Referring to FIGS. 1 to 4, the neural interface (10) according to an embodiment of the present disclosure includes a dual clip design with electrode sections (E1, E2, E3, E4), an electrode pad section (P), and wiring (L) disposed on a substrate (110).

The electrode sections (E1, E2, E3, E4) may be positioned on the upper part of the neural interface (10). The electrode sections (E1, E2, E3, E4) can contact nerves for neural signal recording and stimulation. The upper part of the neural interface 10 may have a dual clip design. Specifically, the upper part of the neural interface 10 may include a first hole (H1), a first region (A1) defined by the first hole (H1), a second hole (H2) disposed within the first region (A1), and a second region (A2) defined by the second hole (H2).

The first hole (H1) may have a “[” shape, allowing the first region (A1) to bend vertically. Similarly, the second region (A2) can also bend vertically due to the second hole (H2).

The electrode section (E1, E2, E3, E4) may contact both the upper and lower parts of the nerve. Specifically, the electrode section (E1, E2, E3, E4) may include a first electrode section (E1), a second electrode section (E2), a third electrode section (E3), and a fourth electrode section (E4). The first electrode section (E1) and the fourth electrode section (E4) may be positioned at the edges of the neural interface (10), where the first electrode section (E1) and the fourth electrode section (E4) can contact the lower part of the nerve.

The second electrode section (E2) and the third electrode section (E3) may be positioned at the center of the neural interface 10, particularly within the second region (A2). The second electrode section (E2) and the third electrode section (E3) positioned at the center of the neural interface 10 can contact the upper part of the nerve through the movement of the first region (A1) and the second region (A2).

Thus, the present disclosure can stably fix the nerve by double clipping.

The electrode pad section (P) may be positioned at the lower part of the neural interface 10 and connected to external electrical signals.

The wiring (L) may electrically connect the electrode section (E1, E2, E3, E4) and the electrode pad section (P).

With reference to FIGS. 2 to 4, the layered structure of the neural interface (10) is explained. The neural interface (10) includes a substrate (110) with a coating layer (112), a first SMP layer (120), an adhesion layer (130), a conductive layer (132), and a second SMP layer (122).

The substrate (110) with the coating layer (112) may be, for example, an Si wafer coated with Al.

The first SMP layer (120) may include a shape memory polymer (SMP) with a glass transition temperature (Tg) close to body temperature. The shape memory polymer may be polymerized using different types of multi-thiol monomers. Specifically, the shape memory polymer is polymerized via a thiol-ene reaction, using three monomers: 1,3,5-triallyl-1,3,5-triazine-2,4,6(1H,3H,5H)-trione (TATATO), trimethylolpropanetris(3-mercaptopropionate) (TMTMP), and tricyclodecanedimethanoldiacrylate (TCMDA).

The adhesion layer (130) is an intermediate layer between the first SMP layer (120) and the conductive layer (132) to enhance the adhesion of the conductive layer (132). For example, the adhesion layer (130) may include materials like chromium (Cr), titanium (Ti), or titanium/titanium nitride (Ti/TiN). The adhesion layer (130) may include various materials that can enhance the adhesion between the first SMP layer (120) and the conductive layer (132).

The conductive layer (132) is placed on the adhesion layer (130) and may transmit electrical signals. For instance, the conductive layer (132) may include gold (Au), platinum, titanium nitride (TiN), molybdenum (Mo), carbon nanotubes (CNT), or conductive polymers like PEDOT:PSS. The conductive layer (132) includes any biocompatible conductive material suitable for neural stimulation and recording.

To further enhance the stability and recording performance of the neural interface (10), additional conductive material may be electrodeposited on the conductive layer (132). Coating with the additional conductive material improves the electrochemical performance of the neural interface (10). For instance, the additional conductive material may be IrO2, gold (Au), carbon nanotubes (CNT), platinum black (Pt-black), titanium nitride (TiN), conductive polymers including PEDOT:PSS, molybdenum (Mo), or etc. So long as it is lower in resistance and higher in charge transfer capability than the material constituting the conductive layer (132), any conductive material may be used as the additionally improved conductive material.

The adhesion layer (130) and the conductive layer (132) may be patterned to form the electrode section (E1, E2, E3, E4), the electrode pad section (P), and the wiring (L) as shown in FIG. 1.

The second SMP layer (122) may include the same material as the first SMP layer (120) and be positioned on the topmost surface of the neural interface (10). The second SMP layer (122) may be patterned to expose the electrode section (E1, E2, E3, E4) and the electrode pad section (P). Also, the second SMP layer (122) may be patterned to form the first hole (H1) and the second hole (H2).

The total thickness of the neural interface of the present disclosure may range from 30 nm to 50 nm. This thin thickness allows the neural interface to be easily implanted.

Turing to FIG. 5, an injection process of the neural interface is described.

For implantation, the neural interface is made soft at a sufficiently high temperature (e.g., above 50° C.). The neural interface is then deformed into a shape that is easy to insert and morphologically fixed at 0° C. Specifically, the first region (A1) and the second region (A2) may each be moved to create a space for nerve insertion and then fixed. After implanting the neural interface into the nerve (N), the shape is restored by spraying saline of the body temperature or similar temperature. Through this process, the neural interface can be easily inserted and fixed to the peripheral nerve (N). Minimizing contact between the neural interface and the neural tissue during the implantation process is crucial to reduce damage on the neural interface and nerve. The present disclosure utilizes the shape memory effect to minimize contact through a self-implantation method.

The neural interface (10) of the present disclosure can be easily inserted into very small nerves through the shape memory effect of the shape memory polymer. Additionally, the low glass transition temperature (44° C.; dry state) allows shape deformation through the shape memory effect without stressing biological tissues, facilitating easy implantation and minimizing damage to both the neural interface and the neural tissue. Furthermore, the neural interface exhibits a Young's modulus of 400 MPa at 37° C., which is softer than conventional polyimide substrates (2.5 GPa).

The neural interface of the present disclosure meets various conditions such as biocompatibility, thinness, lower Young's modulus, and simplified fabricating through photolithography processes for micro-patterning. Moreover, utilizing the characteristics of shape memory polymers, the neural interface can be easily implanted into small nerves with a 3D structure. The neural interface of the present disclosure is expected to find applications not only in neural modulation for disease treatment but also in recording neural signals for bionic limb operation and nerve stimulation applied techniques for user's sensory feedback.

Referring to FIG. 6, a method is elucidated for fabricating a neural interface according to the present disclosure.

With reference to FIG. 6, the method for fabricating a neural interface according to the present disclosure includes the following steps of: synthesizing a shape memory polymer (S110); first coating the SMP onto a substrate (S120); forming a photoresist layer (S130); masking (S140); sputtering an electrode material (S150); patterning through a photolithography process (S160); second coating the shape memory polymer (S170); and etching the shape memory polymer (S180).

In step (S110) of synthesizing a shape memory polymer, the shape memory polymer can be synthesized by polymerizing 1,3,5-triallyl-1,3,5-triazine-2,4,6(1H, 3H, 5H)-trione (TATATO), trimethylolpropanetris(3-mercaptopropionate) (TMTMP), and tricyclodecanedimethanoldiacrylate (TCMDA). In step (S110) of synthesizing a shape memory polymer, these three monomers can be polymerized via a thiol-ene reaction. The polymerization of TATATO, TMTMP, and TCMDA affects the molecular weight and chain formation of the polymer, which can alter the glass transition temperature (Tg).

In this process, 2,2-dimethoxy-2-phenyl-acetophenone (DMPA) can be used as a photoinitiator. By controlling the ultraviolet (UV) exposure time involved in the polymerization process with the photoinitiator, the chain formation of the polymer can be adjusted to lower the glass transition temperature of the resulting shape memory polymer. For example, in one embodiment of the present disclosure, the UV exposure time may be between 20 and 40 minutes, preferably 30 minutes, which thus results in a softer characteristic at the same temperature and enables the utilization of the shape memory effect at physiological temperatures.

Next, in step (S120) of first coating the shape memory polymer onto a substrate, the shape memory polymer synthesized previously may be coated onto the substrate. The substrate may be, for example, a silicon wafer coated with an aluminum thin film. The shape memory polymer may be spin-coated onto the substrate. The spin-coating process facilitates easy control of the coating thickness and allows for a uniform deposition.

In step (S130) of forming a photoresist layer, a photoresist layer may be deposited on the shape memory polymer.

In step (S140) of masking, the photoresist is patterned using a chrome hard mask and a mask aligner, considering the patterns for the electrode section, electrode pad section, and wiring.

In step (S150) of sputtering an electrode material, the entire surface is sputtered with the electrode material. Both the adhesion material and the conductive material are sputtered. For example, chromium (Cr) may be sputtered as an adhesion material, and gold (Au) may be sputtered as a conductive material. However, the embodiments are not limited to these materials, and various biocompatible conductive materials may be used as adhesion and conductive materials.

When depositing 200 nm of gold (Au) by sputtering, process conditions may be set to be 3 mTorr to 10 mTorr for the chamber pressure, 150 W to 250 W for the applied power, 2 to 5 minutes for the exposure time, and 10 sccm to 30 sccm for argon gas injection rate. Preferably, for 200 nm gold (Au) deposition, the sputtering may be carried out at a chamber pressure of 5 mTorr, an applied power of 200 W, and an exposure time of 3 minutes and 30 seconds, with argon gas injected at a rate of 20 sccm.

When depositing 50 nm of chromium (Cr) by sputtering, process conditions may be set to be 10 mTorr to 30 mTorr for the chamber pressure, 150 W to 250 W for the applied power, 1 to 3 minutes for the exposure time, and 30 sccm to 70 sccm for argon gas injection rate. Preferably, for 50 nm gold (Au) deposition, the sputtering may be carried out at a chamber pressure of 20 mTorr, an applied power of 200 W, and an exposure time of 2 minutes and 10 seconds, with argon gas injected at a rate of 50 sccm.

In step (S160) of patterning through photolithograph, the photoresist may be removed to pattern the adhesion layer and the conductive layer. This step involves patterning the adhesion layer and the conductive layer into the electrode section, electrode pad section, and wiring patterns.

In step S170 of second coating the shape memory polymer, the shape memory polymer is coated using a method identical or similar to the first coating step (S120). Specifically, the shape memory polymer is spin-coated over the adhesion layer and the conductive layer.

In step (S180) of etching the shape memory polymer, the shape memory polymer is etched into the desired shape. That is, the shape memory polymer is etched such that the electrode section and the electrode pad section are exposed. Additionally, the first hole and the second hole may be formed to achieve the dual clip design as described in the foregoing. In this regard, when a chrome hard mask is patterned on the shape memory polymer, followed by reactive ion etching (RIE) with oxygen plasma. Specifically, the patterning conditions may be set to be 110 sccm to 130 sccm for oxygen (O₂) flow rate, 10 mTorr to 30 mTorr for chamber pressure, and 1 minute to 5 minutes for purge time. Preferably, the patterning may be carried out at a chamber pressure of 20 mTorr for an operation time of 20 min×8 times with a purge time of 3 min while oxygen (O₂) was flowed at a rate of 120 sccm. Thereafter, aluminum etchant and chrome etchant may be used to remove the sacrificial aluminum thin film and the chrome hard mask, resulting in the final neural interface

The present disclosure uses sputtering and photolithography processes to directly deposit and pattern conductive materials. This eliminates the need for pre-metal layer processes (stamping), allowing the neural interface to be fabricated using a single wafer. Furthermore, the use of sputtering instead of the conventional E-beam process for metal deposition reduces the process cost and thus can be more suitable for mass production.

A better understanding of the present disclosure may be obtained through the following examples, which are set forth illustrate, but are not to be construed to limit, the present disclosure.

### Examples

1,3,5-Triallyl-1,3,5-triazine-2,4,6(1H,3H,5H)-trione (TATATO) (Sigma-Aldrich, Seoul, Korea), trimethylolpropanetris(3-mercaptopropionate) (TMTMP) (Sigma-Aldrich, Seoul, Korea), tricyclodecanedimethanoldiacrylate (TCMDA) (Sigma-Aldrich, Seoul, Korea), and 2,2-dimethoxy-2-phenyl-acetophenone (DMPA) (Sigma-Aldrich, Seoul, Korea) were used to synthesize the SMP. The synthesis process was conducted in a class 1000 cleanroom. The SMP was synthesized from a mixture of stoichiometric amounts of TATATO and TMTMP, 31 mol % TCMDA, and 0.1 wt % DMPA as a photoinitiator. The solution was mixed using a paste mixer (AR-100, THINKY, Laguna Hills, CA, USA) to obtain a homogeneous solution.

Using a spin coater (SPIN-1200T, MIDAS, Seongnam, Korea), the solution was first applied to an Al-coated Si wafer at 800 rpm for 25 seconds to achieve a thickness of 20 μm. The Al thin film was used as a sacrificial layer. The coating was then cured under 365 nm UV light for 1 hour and dried in a 120° C. vacuum oven for 24 hours.

Next, a photoresist (DNR-L300-40, Dongin-Semichem, Seoul, Korea) was formed and patterned using a chrome hard mask and a mask aligner. Chromium (Cr) and gold (Au) thin films, serving as the adhesion and conductive layers respectively, were deposited to thicknesses of 50 nm and 200 nm using sputtering equipment. The photoresist was then removed to pattern the adhesion and conductive layers.

A second coating and curing of the SMP, with a thickness of 20 μm, was performed in the same manner as the first. A 200 nm chrome hard mask was patterned on the SMP using the same method as in the foregoing adhesive layer and conductive layer. The SMP was etched into the desired shape and micropatterned using reactive ion etching (RIE) with oxygen plasma. The sacrificial aluminum thin film and the chrome hard mask were removed using the aluminum etchant HCl 2M solution and chromium etchant (Sigma-Aldrich, Seoul, Korea), respectively.

Additionally, to enhance the stability and recording performance of the neural interface, a version of the neural interface with IrO₂ electrodeposition on the conductive layer was also prepared.

### Experimental Example 1: Characterization of Mechanical Properties

The mechanical properties of the SMP substrate were analyzed using dynamic mechanical analysis (DMA). A dynamic mechanical analyzer (DMA Q800, TA Instruments, New Castle, DE, USA) was used to measure the storage modulus (E′) and the loss factor (tan δ). Samples were prepared by laser cutting a 20 μm thick SMP film into rectangular shapes of 40 mm×6 mm. Measurements were conducted at 0.2N preload, 0.275% strain, and 1 Hz, with a temperature change rate of 2° C. min⁻¹ from −10° C. to 80° C.

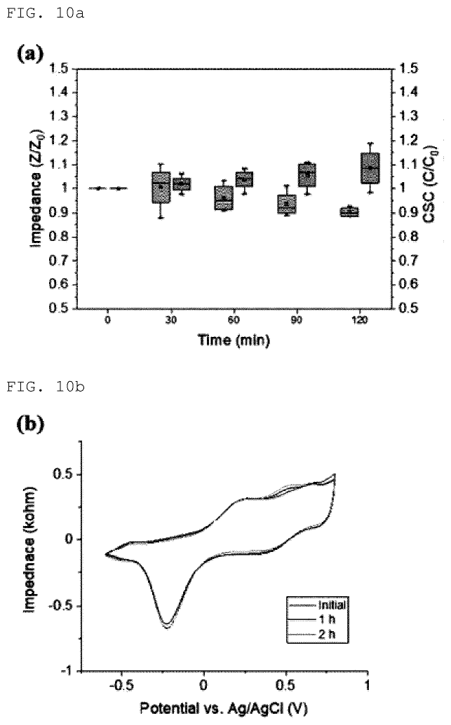

FIG. 8(a) shows the storage modulus of the SMP. At room temperature (22° C.), the storage modulus of the SMP was 1.4 GPa. This level of stiffness allowed for complex micropatterning through photolithography. The SMP began to soften rapidly from 30° C. due to the polymer's properties, with a Young's modulus of 400 MPa at body temperature (37° C.). FIG. 8(b) indicates that the glass transition temperature was 44° C.

### Experimental Example 2: Characterization of Electrochemical Properties

To analyze the performance of the neural interface, electrochemical impedance spectroscopy (EIS) tests were performed using a multichannel potentiostat (Ivium-n-Stat, IVIUM Technology). A three-electrode setup was used with an Ag/AgCl reference electrode and a platinum (Pt) counter electrode. Phosphate buffered saline (PBS) was used as the medium, with a scan rate of 50 mV/s and a frequency range of 1 Hz to 100 kHz.

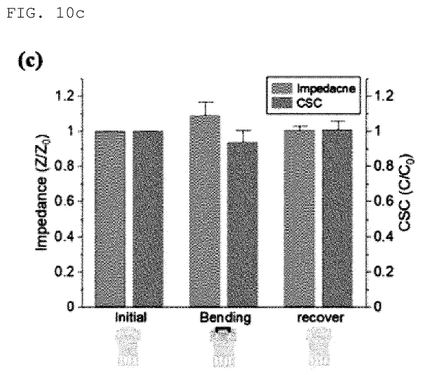

The impedance and charge storage capacity (CSC) of the Au-deposited neural interface and the IrO₂-coated neural interface were compared. The impedance of the neural interface was measured over the frequency range of 1 Hz to 100 kHz. FIG. 9(a) shows that the impedance at 1 kHz for the uncoated neural interface and the IrO2-coated neural interface were 30.74 kΩ and 2.114 kΩ, respectively.

The CSC was measured using cyclic voltammetry (CV) with an applied voltage range of −0.6 V to 0.8 V and a scan rate of 50 mV/s. FIG. 9(b) shows that the CSC for the uncoated neural interface and the IrO₂-coated neural interface were 0.19 mC/cm² and 14.59 mC/cm², respectively.

In summary, the IrO₂ coating reduced impedance by a factor of 14.54 and increased CSC by a factor of 76.77.

### Experimental Example 3: Stability Confirmation

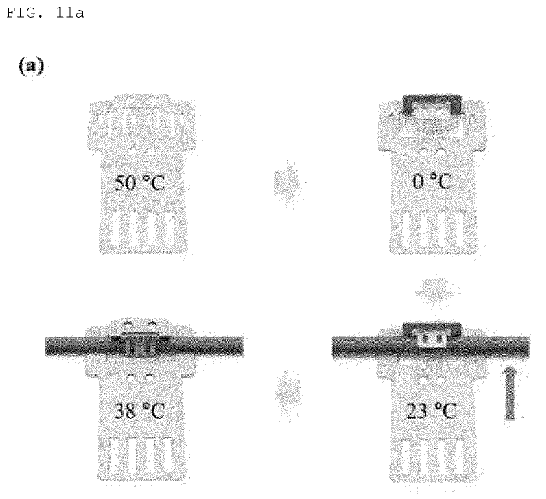

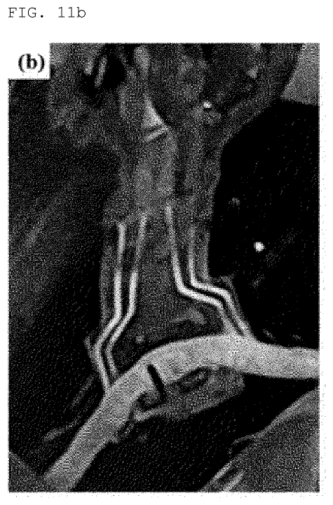

To confirm the stable electrodeposition of IrO2 coating, a stability test was conducted. A continuous voltage was applied to the neural interface for 2 hours in PBS. Using the same CV measurement method as in Experimental Example 2 with the potentiostat, a biphasic triangular pulse with a maximum amplitude of 0.8V and a minimum amplitude of −0.6V was applied once per second. Impedance and CSC were measured at 30-minute intervals to monitor the electrode condition, as shown in FIG. 10(a). After 2 hours, the impedance of the neural interface decreased by 9.64%, and the CSC increased by 8.45%.

As shown in FIG. 10(b), the state of the coated electrode surface was indirectly confirmed by fitting the CV curves over time. There were no significant changes observed in the CV curves or their magnitudes, indicating that the performance of the neural interface did not degrade over prolonged voltage application.

Another stability test was performed to determine the effect of the bending process during the implantation of the neural interface. Impedance and CSC were measured in three states: the initial state (Initial), the bent state (Bending) after being deformed into a hook shape through the bending process, and the recovered state (Recover) after restoring the shape using the shape memory effect. As shown in FIG. 10(c), bending stress, which is generated in a bent state, caused an 8.84% increase in impedance and a 6.47% decrease in CSC. After recovery at 38° C., impedance increased by 0.39% and CSC increased by 0.65% compared to the initial state. The bending stress impacted the performance of the neural interface during the bending process, but the performance change after recovery was less than 1%, indicating that there was no significant impact on stimulation and recording performance.

### Experimental Example 4: Functional Effect after Implantation

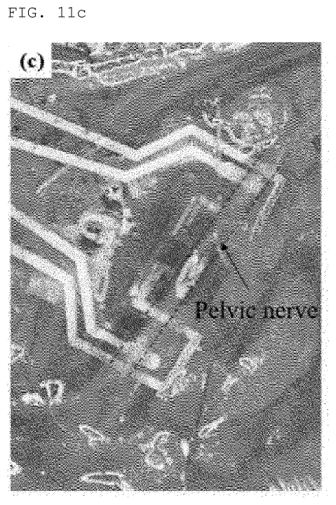

As shown in FIGS. 11(b) and (c), the fabricated neural interface was implanted in the common peroneal nerve (CPN) and the pelvic nerve (PN) and analyzed for functional effect. A reshape procedure was conducted to facilitate implantation. As shown in FIG. 11(a), the neural interface was heated on a 50° C. hot plate to minimize damage. The neural interface was then deformed into a hook shape and fixed at 0° C. The hook-shaped neural interface was fixed to the nerve, and 38° C. phosphate buffered saline (PBS) was sprayed on the neural interface. Due to the shape memory effect, the neural interface self-clipped and fixed itself to the nerve.

### Experimental Example 5: Stimulation and Recording Test

Stimulation and recording were performed at the acute level in Sprague Dawley (SD) rats. Stimulation tests were conducted using the neural interface to confirm neural stimulation. An RHD 2216 system (Intan Technologies, LA, USA) was used to acquire electromyography (EMG) signals at a sampling rate of 30 kHz. A 60 Hz notch filter and band-pass filters with cutoff frequencies of 0.1 Hz and 2.5 kHz were applied for signal recording. An RHD 2132 system (Intan Technologies, Los Angeles, CA, USA) was used to collect compound nerve action potentials (CNAPs) at a sampling rate of 30 kHz. The same notch and band-pass filters as those used for the RHD 2216 were applied. The acquired signals were processed using MATLAB 2020a software. EMG signals induced from the tibialis anterior (TA) muscle and CNAPs from the common peroneal nerve (CPN) were recorded and averaged 60 times every second to reduce noise.

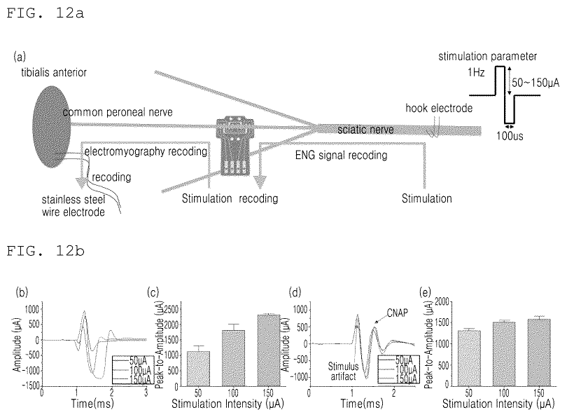

Turning to FIG. 12(a), the nerve was stimulated through the neural interface implanted in the CPN, and muscle signals were recorded using stainless steel wires implanted in the tibialis anterior (TA) muscle. A biphasic waveform of 100 μs pulses was applied at 1 Hz for 1 minute, with current amplitudes varying from 50 μA to 100 μA to 150 μA. Muscle twitches were observed after stimulation. FIG. 12(b) shows the EMG signals processed using the aforementioned signal processing method.

As shown in FIG. 12(c), the peak-to-peak amplitudes of the acquired EMG signals were 1120.4 μA at 50 μA, 1513.8 μA at 100 μA, and 2221.7 μA at 150 μA, confirming that the magnitude of the EMG signals increased with the stimulation intensity.

To verify the recording performance of the neural interface, a recording test was conducted. Compound nerve action potentials (CNAPs) were recorded through the neural interface in the CPN by stimulating the sciatic nerve (SN) with a hook electrode. Electrical stimulation was applied through the hook electrode, with stimulation parameters identical to those used in the stimulation test. Leg tremors were observed due to the stimulation.

Electroneurography (ENG) signals were acquired and processed using the previously mentioned signal processing method. FIG. 12(d) shows the processed ENG signals.

As shown in FIG. 12(e), the peak-to-peak amplitudes of the acquired ENG signals were 1308.2 μA at 50 μA, 1498.1 μA at 100 μA, and 1544.9 μA at 150 μA. These results indicate that the stimulation parameters exceeded the threshold energy, and the amplitude of the evoked CNAPs saturated as the stimulation amplitude increased.

Overall, the results demonstrate that the neural interface made of SMP shows excellent implantation performance due to the shape memory effect and is suitable for use in neural recording and stimulation.

The features, structures, effects and the like described in the above embodiments are included in at least one embodiment of the present invention, and are not necessarily limited to only one embodiment. In addition, the features, structures, effects and the like described in each embodiment may be combined or modified with respect to other embodiments by those skilled in the art. Accordingly, contents related to these combinations and modifications should be construed as being included in the scope of the present invention.

Although the embodiments of the present invention have been described above in detail with reference to the accompanying drawings, it will be apparent to those skilled in the art that various substitutions, modifications, and alterations may be devised within the spirit and scope of the present invention. For example, respective constituent elements, which are concretely described in the embodiments, may be altered in various ways. Differences related to these modifications and applications should be construed as included within the scope of the present disclosure as defined by the appended claims.

### INDUSTRIAL APPLICABILITY

The present disclosure is characterized by a dual clip design implemented utilizing shape memory polymers for a bio-implantable neural interface for recording and stimulating neural signals of peripheral nerves. Particularly, using a sputtering method for metal coating on the shape memory polymer coating layer can reduce the process cost and is suitable for mass production, so that the present disclosure is highly industrially applicable.

1. A neural interface, comprising:
an electrode section for neural stimulation and neural signal recording;
a first region defined by a first hole; and
a second region defined by a second hole disposed within the first region,
wherein the electrode section comprises a first electrode section and a second electrode section, either the first electrode section or the second electrode section being disposed within the second region.

2. The neural interface of claim 1, wherein,
the first electrode section is disposed at an edge of the neural interface, and
the second electrode section is disposed within the second region.

3. The neural interface of claim 1, wherein the first electrode section and the second electrode section are in contact with the upper and lower parts of the nerve, respectively.

4. The neural interface of claim 1, wherein the electrode section comprises:
a substrate;
a shape memory polymer (SMP) layer disposed on the substrate;
an adhesion layer disposed on the SMP layer; and
a conductive layer disposed on the adhesion layer.

5. The neural interface of claim 1, further comprising wiring electrically connected to the electrode section, wherein the wiring comprises:
a substrate;
a first SMP layer disposed on the substrate;
an adhesion layer disposed on the first SMP layer;
a conductive layer disposed on the adhesion layer; and
a second SMP layer disposed on the conductive layer.

6. The neural interface of claim 4, wherein the shape memory polymer is synthesized by polymerizing 1,3,5-triallyl-1,3,5-triazine-2,4,6(1H,3H,5H)-trione (TATATO), trimethylolpropanetris(3-mercaptopropionate) (TMTMP), and tricyclodecanedimethanoldiacrylate (TCMDA).

7. A method for fabricating a neural interface, the method comprising the steps of:
synthesizing a shape memory polymer;
first coating a substrate with the shape memory polymer;
forming a photoresist on the shape memory polymer;
masking the photoresist;
sputtering an electrode material;
patterning the electrode material through a photolithography process;
second coating the shape memory polymer; and
etching the shape memory polymer.

8. The method of claim 7, wherein the step of synthesizing a shape memory polymer is carried out by polymerizing 1,3,5-triallyl-1,3,5-triazine-2,4,6(1H,3H,5H)-trione (TATATO), trimethylolpropanetris(3-mercaptopropionate) (TMTMP), and tricyclodecanedimethanoldiacrylate (TCMDA).

9. The method of claim 7, wherein in the first coating step, the shape memory polymer is spin-coated.

10. The method of claim 7, wherein in the masking step, the photoresist is patterned using a chrome hard mask and a mask aligner.

11. The method of claim 7, wherein in the sputtering step, the adhesion material and the conductive material are formed by sputtering.

12. The method of claim 1, wherein the etching step comprises reactive ion etching using oxygen plasma.
